(19) United States
(12) Patent Application Publication
(10) Pub. No.: US 2008/0102041 A1
(43) Pub. Date: May 1, 2008

(54) Teeth Stain Inhibitor Composition and Methods of Use Thereof

(76) Inventors: John Scott Alden, Sequim, WA (US); Imogen Sue Kohnert, Sequim, WA (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 11/553,602

(22) Filed: Oct. 27, 2006

(51) Int. Cl. A61K 8/31

(52) U.S. Cl. 424/49

(57) ABSTRACT

The invention provides ingestible compositions for reducing or inhibiting teeth stain, and methods for using the same.

### FIELD OF THE INVENTION

This invention relates to ingestible teeth stain reducing or inhibiting compositions and methods for using the same.

### BACKGROUND OF THE INVENTION

The majority of people consider clean, white teeth to be aesthetically very desirable. Teeth with extrinsic stains are objectionable to the general public both on the basis of cosmetic appearance and also socially as an indication of poor oral hygiene. Because the acquired pellicle, which coats the teeth, has a natural tendency to stain, most people will form some unsightly extrinsic stains on their teeth over time. This staining process is promoted by the ingestion of tannin-containing foods and beverages such as coffee, tea, or red wine; the use of tobacco products; and/or exposure to certain cationic substances such as tin, iron, and chlorhexidine.

Unblemished white teeth have long been considered cosmetically desirable. Unfortunately, in the absence of thorough dental cleaning, teeth can become discolored or stained from color-causing substances present in food, beverages, tobacco, and the like, and internal sources such as blood, amalgam-based fillings, and antibiotics (e.g., tetracycline).

The tooth structures that are generally responsible for presenting a stained appearance are enamel, dentin, and the acquired pellicle. Tooth enamel is predominately formed from inorganic material, mostly in the form of hydroxyapatite crystals, and further contain approximately 5% organic material primarily in the form of collagen. In contrast, dentin is composed of about 20% protein including collagen, the balance existing of inorganic material, predominately hydroxyapatite crystals, similar to that found in enamel. The acquired pellicle is a proteinaceous layer present on the surface of tooth enamel which reforms rapidly after an intensive tooth cleaning.

Discoloration of teeth can result from extrinsic and/or intrinsic staining. Extrinsic staining of the acquired pellicle can arise as a result of compounds, such as tannins and other polyphenolic compounds, that have become trapped in and tightly bound to the proteinaceous layer on the surface of the teeth. Discoloration from this type of staining can usually be removed by mechanical methods of tooth cleaning. In contrast, intrinsic staining occurs when the staining compounds penetrate the enamel and even the dentin, or alternatively, such staining arises from sources within the tooth. Discoloration from intrinsic staining is not readily amenable to mechanical methods of tooth cleaning. Chemical methods, which utilize substances that can penetrate into the tooth structure, are usually required to eliminate such discoloration.

Currently, there are a number of methods for removing stains in teeth. These methods are generally based on the use of abrasives, hydrolytic agents or oxidizing agents to break down the staining material. The most widely practiced method for the control of extrinsic stains is daily tooth brushing with dentifrices. However, tooth brushing alone is not capable of completely preventing stain formation. Areas of the dentition, which are commonly missed during tooth brushing, such as the interproximal tooth surfaces and the lingual areas of the anterior teeth, are very prone to stain accumulation. Once the stain has formed, it is very difficult to remove without obtaining a professional dental cleaning.

In fact, most commercial teeth whitening products are based on removal of stains that have already accumulated. While these products serve their intended purposes, it would be desirable to reduce or prevent teeth staining in the first place.

Therefore, there is a need for preventing and/or inhibiting potential stain causing substances from adhering to teeth surfaces.

### SUMMARY OF THE INVENTION

One aspect of the invention provides compositions for reducing, avoiding, and/or prolonging the need for the use of teeth whitening compositions, procedures, or other costly teeth whitening practices that are usually conducted after the stains are present. In some embodiments, compositions of the invention comprise an ingestible teeth whitening or teeth stain inhibitor. In some embodiments, a combination of two or more ingestible ingredients produces a synergistic teeth stain prevention. Without being bound by any theory, it is believed that compositions of the invention inhibit and/or prevent potential stain causing substances from adhering to the teeth when applied to the tooth surface.

In certain embodiments of the invention, compositions of the invention can also comprise a food flavoring.

Another aspect of the invention provides an ingestible oral care composition for inhibiting or reducing teeth stain that consists of: water, preservative, sweetener, phosphoric acid, flavoring agent and xylitol.

As stated above, some embodiments of the invention comprise a composition that is ingestible. In these embodiments, simply drinking the composition of the invention reduces, prevents, and/or inhibits formation of teeth stain. Preferably, the subject swishes and/or swirls the composition of the invention in the subjects mouth in order to increase the effectiveness of reducing, preventing, and/or inhibiting teeth stain formation.

Another aspect of the invention provides, methods for using the same for reducing, preventing and/or inhibiting teeth stain. Typically, methods of the invention comprise drinking a composition of the invention to remove, reduce, prevent, or inhibit formation of extrinsic stains from external surfaces of the teeth.

### DETAILED DESCRIPTION OF THE INVENTION

One aspect of the invention provides compositions for inhibiting the potential stain causing substances from adhering to teeth surfaces. As used herein, the term “inhibiting” refers to reducing, retarding, preventing, and/or inhibiting teeth stain from forming in the first instance. In one embodiment, the invention provides an ingestible oral care composition for inhibiting teeth stain comprising an ingestible teeth stain inhibiting agent.

Exemplary teeth stain inhibiting agents include, but are not limited to, food grade hydrogen peroxide, carbamide peroxide, sodium hexametaphosphate, xylitol, baking soda, anthium dioxide, casein and/or it's hydrolysate solution(s), lactoperoxidase inhibitory enzymes (e.g., lysozyme), oxygenating agents, natural antibacterial enzymes, and processed components from the morinda citrifolia L plant. In some embodiments, teeth stain inhibiting or reducing agent is an ingestible whitening and stain inhibitive dentifrice agents.

The total amount of teeth stain inhibiting agent is present in compositions of the invention at a tooth stain preventive effective concentration. A tooth stain preventative effective concentration is preferably less than 10% (or about 10%), more preferably less than 5% (or about 5%), and most preferably from 0.05% (or about 0.05%) to 1% (or about 1%). Percentages referred to herein are in weight-volume (w/v) units for liquid embodiments and weight-weight (w/w) units for semi-solid and solid embodiments.

Compositions of the invention can be in a liquid or a solid form. In some instances, compositions of the invention is in a liquid form. In this manner, one can simply drink the composition of the invention after eating food that can stain teeth. Preferably, the subject places a portion of the liquid composition of the invention in the subject's mouth, swishes and/or swirls the composition for a few seconds before swallowing. It should be appreciated that while compositions of the invention is ingestible composition, the subject need not swallow the liquid after swishing and/or swirling it in the subject's mouth. The subject can expel, e.g., spit-out, the composition after swishing and/or swirling.

Compositions of the invention can also include an ingredient selected from emulsifier, alcohol, sweetener, thickening agent, surfactant, suspending agent, astringent and toning drug extract, flavor correctant, deodorizing agent, preservative, flavoring buffer, whitening agent, inflammation inhibiting substance, antimicrobial agent, pH buffer, and a combination thereof.

Compositions of the invention can also include an essential oil. Essential oils are volatile aromatic oils that are synthetic or are derived from plants by distillation, expression or extraction. Essential oils usually carry the odor or flavor of the plant from which they are obtained. If used in the dentifrice compositions of this invention, essential oils may provide anti-gingivitis activity. Some of these essential oils also act as flavoring agents. The essential oils of this invention include, but are not limited to, thymol, menthol, methyl salicylate (wintergreen oil), and eucalyptol.

Flavoring agents useful for the invention are any food grade or pharmaceutically acceptable, ingestible flavoring agent known to one skilled in the art. Typically, the flavoring agent comprises natural flavoring oils, including those selected from the group consisting of oil of peppermint, oil of wintergreen, oil of spearmint, clove bud oil, parsley oil, eucalyptus oil, cocoa, orange and the like. Combinations of oils can also be used. The flavoring agents may comprise compounds selected from the group consisting of menthol, menthane, anethole, methyl salicylate, eucalyptol, cassia, 1-methyl acetate, sage, eugenol, oxanone, alpha-irisone, marjoram, lemon, orange, propenyl guaethol acetyl, cinnamon, vanilla, thymol, linalool, cinnamaldehyde glycerol acetal and the like, and combinations thereof The flavoring agent may comprise combinations of natural flavoring oils and other flavoring agents such as the compounds identified above. Also, the flavoring agent may comprise cooling agents such as menthol, N-substituted p-menthane-3-carboxamides (such as N-ethyl p-menthane-3-carboxamide), 3,1-methoxy propane 1,2-diol and the like, or combinations thereof.

Any food grade and/or pharmaceutically acceptable sweetener maybe used in the ingestible tooth stain preventative compositions including, but not limited to, xylitol, erythritol, sorbitol, maltitol, mannitol, isomalt, saccharin, fructose, saccharin salts, thaumatin, aspartame, D-tryptophan, dihydrochalcones, acesulfame, and cyclamate salts (e.g., sodium cyclamate). It should be noted that some of the ingredients have more than one purpose. For example, xylitol is both sweetener and stain inhibitor/remover.

In addition to flavoring and sweetening agents, coolants, salivating agents, warming agents, and numbing agents can be used as optional ingredients in compositions of the invention. Exemplary warming agents include capsicum and nicotinate esters, such as benzyl nicotinate. Exemplary numbing agents include benzocaine, lidocaine, clove bud oil, and ethanol. These agents can be present in the compositions at a level of from about 0.001% to about 10%, preferably from about 0.1% to about 1%, by weight of the composition.

The coolant can be any of a wide variety of materials. Included among such materials are carboxamides, menthol, ketals, and diols. Exemplary coolants in the present compositions include paramenthan carboxyamide agents such as N-ethyl-p-menthan-3-carboxamide, known commercially as “WS-3” and N,2,3-trimethyl-2-isopropylbutanamide, known as “WS-23”. Other known coolants include menthol, 3-1-menthoxypropane-1,2-di-ol, menthone glycerol acetal, and menthyl lactate. The terms menthol and menthyl as used herein include dextro- and levorotatory isomers of these compounds and racemic mixtures thereof

In some embodiments, compositions of the invention mask oral malodor, attack the chemicals that bring about the oral malodor, kill or inhibit growth of the bacteria in the mouth that cause breath malodor or halitosis, attack tartar, remove dirt from the teeth and mouth and/or whiten teeth.

In one example, a mixture of 92 gallons of water, 250 g of preservative (e.g., sodium benzoate), 91 g of sweetener (e.g., potassium sorbate), 39 pounds of xylitol, 100 mL of phosphoric acid, and 77 fluid ounces of peppermint flavoring was mixed to provide ingestible teeth stain inhibitor or teeth stain reducing agent.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An ingestible oral care composition for inhibiting teeth stain comprising an ingestible teeth stain inhibiting or teeth stain reducing agent.

2. The composition of claim 1, wherein said composition is a liquid.

3. The composition of claim 1 further comprising an ingredient selected from the group consisting of emulsifier, alcohol, sweetener, thickening agent, surfactant, suspending agent, astringent and toning drug extract, flavor correctant, deodorizing agent, preservative, flavoring buffer, whitening agent, inflammation inhibiting substance, antimicrobial agent, pH buffer, and a combination thereof.

4. The composition of claim 1, wherein said ingestible teeth stain inhibiting or teeth stain reducing agent comprises xylitol.

5. An ingestible oral care composition for inhibiting or reducing teeth stain, said composition consisting of: water, preservative, sweetener, phosphoric acid, flavoring agent and xylitol.
